(19) United States
(12) Patent Application Publication
(10) Pub. No.: US 2022/0015716 A1
(43) Pub. Date: Jan. 20, 2022

(54) ELECTRONIC DEVICE FOR SCREENING RISK DEGREE OF OBSTRUCTIVE SLEEP APNEA, AND OPERATION METHOD THEREFOR

(71) Applicant: Samsung Electronics Co., Ltd., Gyeonggi-do (KR)

(72) Inventors: Wonkyu LEE, Gyeonggi-do (KR); Dawoon JUNG, Gyeonggi-do (KR); Minsoo KIM, Gyeonggi-do (KR); Hyunsu HONG, Gyeonggi-do (KR)

(21) Appl. No.: 17/485,767

(22) Filed: Sep. 27, 2021

Related U.S. Application Data
(63) Continuation of application No. PCT/KR2020/003674, filed on Mar. 18, 2020.

(30) Foreign Application Priority Data
Mar. 25, 2019 (KR) 10-2019-0033358

(51) Int. Cl. A61B 5/00; A61B 5/024; A61B 5/11; A61B 5/0205; A61B 5/08; G16H 40/67; G16H 50/30; G16H 10/60; G16H 50/70; G01P 13/00

(52) U.S. Cl. CPC A61B 5/7275; A61B 5/681; A61B 5/4818; A61B 5/742; A61B 5/11; A61B 5/0205; A61B 5/0816; A61B 5/7221; A61B 5/4842; A61B 5/02405; G16H 40/67; G16H 50/30; G16H 10/60; G16H 50/70; G01P 13/00; A61B 2562/0219; A61B 5/02416

(57) ABSTRACT

An electronic device and method are disclosed for sleep apnea detection. The electronic device includes a display, a photoplethysmogram (PPG) sensor, a memory and a processor. The processor implements the method, including: receiving, for a user, first data from a photoplethysmogram (PPG) sensor, the first data including a pulse signal measured during a first period, and associated with a heartbeat and respiration of the user, determining a first parameter associated with a high-frequency component of the pulse signal, and a second parameter associated with a low-frequency component of the pulse signal, based on at least a portion of the first data, determining fluctuation ranges for the high-frequency component and the low-frequency component, determining whether at least the determined fluctuation ranges are indicative of obstructive sleep apnea (OSA), and displaying information associated with the determination of whether the at least the determined fluctuation ranges are indicative of OSA through the display.

### CROSS-REFERENCE TO RELATED APPLICATION(S)

This application is continuation of International Application No. PCT/KR2020/003674, filed on Mar. 18, 2020, which claims priority to Korean Patent Application No. 10-2019-0033358 filed on Mar. 25, 2019 in the Korean Intellectual Property Office, the disclosures of which are herein incorporated by reference.

### TECHNICAL FIELD

Embodiments of the disclosure relate to an electronic device and method for screening for obstructive sleep apnea.

### BACKGROUND ART

Obstructive sleep apnea is evaluated using nocturnal polysomnography (NPSG). According to polysomnography, abnormal respiration may be detected by continuously monitoring biometrics during sleep, including respiration airflow, respiratory movement of the chest and abdomen, and blood oxygen saturation, using a plurality of sensors and other related equipment.

However, performing polysomnography to determine the severity of the obstructive sleep apnea typically requires a patient to sleep overnight under supervision of a healthcare professional in a facility equipped with the requisite equipment and environments. This incurs high testing costs, thereby making it difficult to access polysomnography. As a result, in society, there may be higher incidence of undiagnosed obstructive sleep apnea.

### Technical Problem

With the emergence of various types of wearable electronic devices and the expansion of the platform service markets, healthcare is receiving much attention as an application field for these developing technologies. A wearable electronic device that is mounted on a wrist may be adapted for usage in healthcare. For example, a motion sensor and a photoplethysmogram (PPG) sensor may be embedded in a wrist-mounted wearable electronic device, and the sensors may enable real-time detection of behavior, posture and heart rate (HR) information.

Various embodiments of the disclosure provide an electronic device that stepwise informs a risk degree of obstructive sleep apnea, using biometric information of a user's body, as measured by a photoplethysmogram (PPG) sensor and a motion sensor.

### Technical Solution

An electronic device according to an embodiment of the disclosure may include a housing, a display visible through a first portion of the housing, a photoplethysmogram (PPG) sensor exposed through a second portion of the housing, a memory disposed within the housing, and a processor disposed within the housing and operatively connected with the memory, the display and the PPG sensor. The memory stores instructions executable by the processor to: receive, for a user, first data from the PPG sensor, the first data including a pulse signal measured during a first period, and associated with a heartbeat and respiration of the user, determine a first parameter associated with a high-frequency component of the pulse signal, and a second parameter associated with a low-frequency component of the pulse signal, based on at least a portion of the first data, determine fluctuation ranges for the high-frequency component of the first parameter and the low-frequency component of the second parameter, determine whether at least the determined fluctuation ranges are indicative of obstructive sleep apnea (OSA), and provide information associated with the determination of whether the at least the determination fluctuation ranges are indicative of OSA through the display.

An electronic device according to an embodiment of the disclosure may include a housing, a display viewable through a first portion of the housing, a photoplethysmogram (PPG) sensor exposed through a second portion of the housing, a memory disposed within the housing, and a processor disposed within the housing and operatively connected with the memory, the display and the PPG sensor, wherein the memory stores instructions that, when executed, cause the processor to: receive, from the PPG sensor, first data including a heart rate (HR) data of a user measured during a first period, determine a parameter associated with a rate-of-change pattern included in the HR data, based on at least a portion of the first data, determine whether at least a portion of the determined parameter is indicative of obstructive sleep apnea (OSA), and provide information associated with the determination whether the at least the portion of the determined parameter is indicative of OSA through the display.

### Advantageous Effects

According to embodiment of the disclosure, a risk degree of obstructive sleep apnea may be stepwise informed by using biometric information of a user, which is measured by a PPG sensor and a motion sensor.

Besides, a variety of effects directly or indirectly understood through this disclosure may be provided.

### DESCRIPTION OF DRAWINGS

FIG. 1 is a block diagram of an electronic device in a network environment according to certain embodiments.

FIG. 2 is a front perspective view of a mobile electronic device according to an embodiment.

FIG. 3 is a rear perspective view of the electronic device of FIG. 2.

FIG. 4 is an exploded perspective view of an electronic device of FIG. 2.

FIG. 5 is a block diagram illustrating an electronic device according to an embodiment of the disclosure.

FIG. 6 is a flowchart illustrating an obstructive sleep apnea screening method of an electronic device according to an embodiment of the disclosure.

FIG. 7 is a flowchart illustrating an obstructive sleep apnea screening method of an electronic device according to certain embodiments of the disclosure.

FIG. 8 is a flowchart illustrating an example of a method in which an electronic device performs a first screening operation in FIG. 7.

FIG. 9A is a flowchart illustrating an example of a method in which an electronic device performs a second screening operation in FIG. 7.

FIG. 9B is a graph illustrating a normal state and an obstructive sleep apnea risk state according to an embodiment.

FIG. 10A is a flowchart illustrating an example of a method in which an electronic device performs a third screening operation in FIG. 7.

FIG. 10B is a graph for determining a result of a breath-holding test according to an embodiment.

FIG. 10C is a graph for determining a result of an orthostatic test according to an embodiment.

FIG. 11 is a view illustrating a user interface in a breath-holding test of an electronic device according to an embodiment.

FIG. 12 is a view illustrating a user interface associated with reexamination in a breath-holding test of an electronic device according to an embodiment.

FIG. 13 is a view illustrating a user interface in an orthostatic test of an electronic device according to an embodiment.

FIG. 14 is a view illustrating a user interface associated with reexamination in an orthostatic test of an electronic device according to an embodiment.

FIG. 15 is a diagram illustrating an electronic device in a network environment according to certain embodiments of the disclosure.

FIG. 16 is a flowchart illustrating an obstructive sleep apnea screening method of an electronic device in a network environment according to certain embodiments of the disclosure.

FIG. 17 is a view illustrating a user interface associated with posture adjustment in an orthostatic test of an electronic device according to an embodiment.

FIG. 18 is a flowchart illustrating a method in which an electronic device provides a result of determining obstructive sleep apnea, according to certain embodiments of the disclosure.

With regard to description of drawings, the same or similar components will be marked by the same or similar reference signs.

### DETAILED DESCRIPTION

Hereinafter, certain embodiments of the disclosure will be described with reference to the accompanying drawings. However, those of ordinary skill in the art will recognize that modification, equivalent, and/or alternative on the certain embodiments described herein can be variously made without departing from the disclosure.

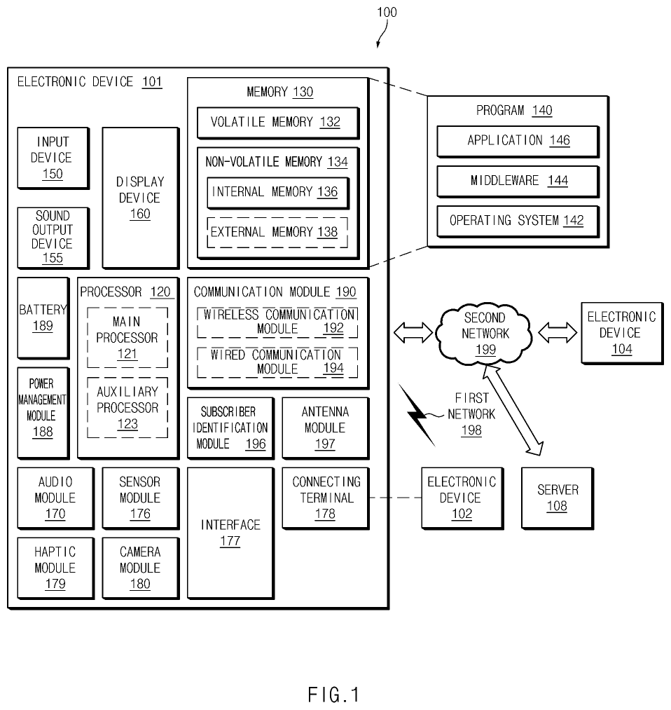

FIG. 1 is a block diagram illustrating an electronic device 101 in a network environment 100 according to certain embodiments. Referring to FIG. 1, the electronic device 101 in the network environment 100 may communicate with an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input device 150, a sound output device 155, a display device 160, an audio module 170, a sensor module 176, an interface 177, a haptic module 179, a camera module 180, a power management module 188, a battery 189, a communication module 190, a subscriber identification module (SIM) 196, or an antenna module 197. In some embodiments, at least one (e.g., the display device 160 or the camera module 180) of the components may be omitted from the electronic device 101, or one or more other components may be added in the electronic device 101. In some embodiments, some of the components may be implemented as single integrated circuitry. For example, the sensor module 176 (e.g., a fingerprint sensor, an iris sensor, or an illuminance sensor) may be implemented as embedded in the display device 160 (e.g., a display).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may load a command or data received from another component (e.g., the sensor module 176 or the communication module 190) in volatile memory 132, process the command or the data stored in the volatile memory 132, and store resulting data in non-volatile memory 134 (which may include internal memory 136 and external memory 138). According to an embodiment, the processor 120 may include a main processor 121 (e.g., a central processing unit (CPU) or an application processor (AP)), and an auxiliary processor 123 (e.g., a graphics processing unit (GPU), an image signal processor (ISP), a sensor hub processor, or a communication processor (CP)) that is operable independently from, or in conjunction with, the main processor 121. Additionally or alternatively, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display device 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input device 150 may receive a command or data to be used by other component (e.g., the processor 120) of the electronic device 101, from the outside (e.g., a user) of the electronic device 101. The input device 150 may include, for example, a microphone, a mouse, a keyboard, or a digital pen (e.g., a stylus pen).

The sound output device 155 may output sound signals to the outside of the electronic device 101. The sound output device 155 may include, for example, a speaker or a receiver. The speaker may be used for general purposes, such as playing multimedia or playing record, and the receiver may be used for an incoming calls. According to an embodiment, the receiver may be implemented as separate from, or as part of the speaker.

The display device 160 may visually provide information to the outside (e.g., a user) of the electronic device 101. The display device 160 may include, for example, a display, a hologram device, or a projector and control circuitry to control a corresponding one of the display, hologram device, and projector. According to an embodiment, the display device 160 may include touch circuitry adapted to detect a touch, or sensor circuitry (e.g., a pressure sensor) adapted to measure the intensity of force incurred by the touch.

The audio module 170 may convert a sound into an electrical signal and vice versa. According to an embodiment, the audio module 170 may obtain the sound via the input device 150, or output the sound via the sound output device 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a cellular network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The antenna module 197 may transmit or receive a signal or power to or from the outside (e.g., the external electronic device) of the electronic device 101. According to an embodiment, the antenna module 197 may include an antenna including a radiating element implemented using a conductive material or a conductive pattern formed in or on a substrate (e.g., PCB). According to an embodiment, the antenna module 197 may include a plurality of antennas. In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 and 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, or client-server computing technology may be used, for example.

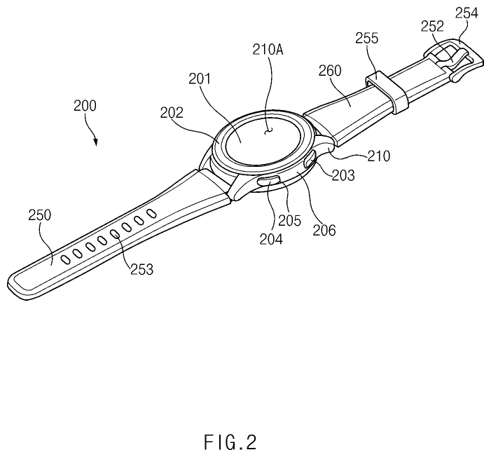

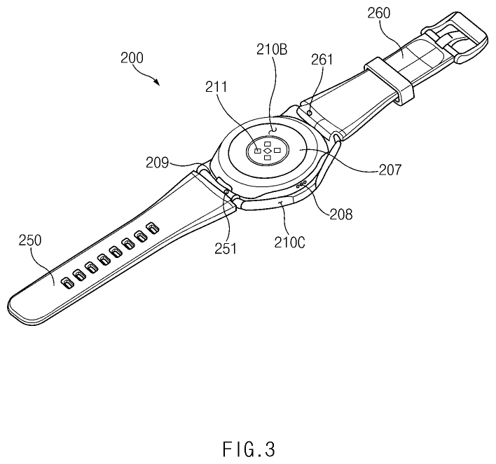

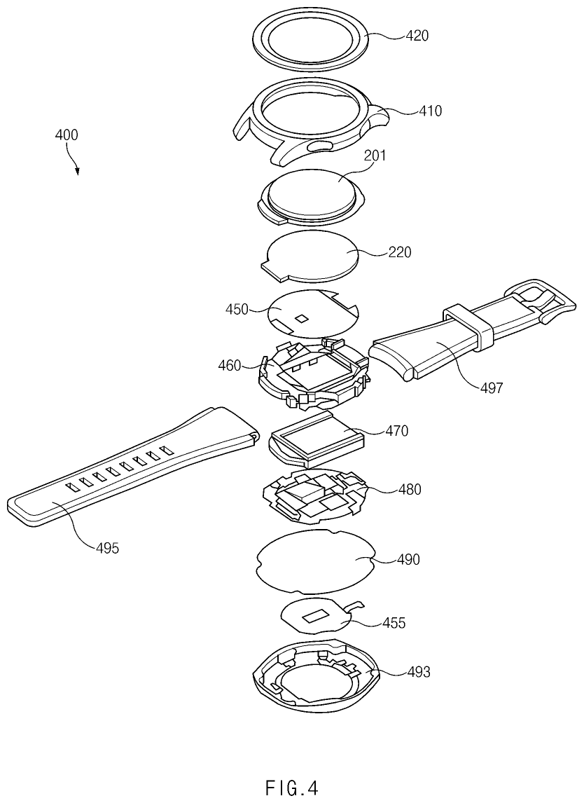

FIG. 2 is a front perspective view of a mobile electronic device according to an embodiment. FIG. 3 is a rear perspective view of an electronic device of FIG. 2. FIG. 4 is an exploded perspective view of an electronic device of FIG. 2.

Referring to FIGS. 2 and 3, an electronic device 200 (e.g., the electronic device 101) according to an embodiment may include a housing 210 including a first surface (or a front surface) 210A, a second surface (or a back surface) 210B, and a side surface 210C surrounding a space between the first surface 210A and the second surface 210B, and binding members 250 and 260 configured to bind the electronic device 200 to a part of a body (e.g., a wrist, an ankle, or the like) of a user so as to be removable. In another embodiment (not illustrated), the housing (210) may be referred to as a “structure” that forms a part of the first surface 210A, the second surface 210B, and the side surface 210C of FIG. 2. According to an embodiment, the first surface 210A may be implemented with a front plate 201 (e.g., a glass plate including various coating layers, or a polymer plate), at least a portion of which is substantially transparent. The second surface 210B may be formed by a back plate 207 that is substantially opaque. For example, the back plate 207 may be formed of a coated or colored glass, a ceramic, a polymer, a metal (e.g., aluminum, stainless steel (STS), or magnesium), or a combination of at least two of the materials. The side surface 210C may be coupled to the front plate 201 and the back plate 207, and may be formed by a side bezel structure (or a “side member”) 206 including a metal and/or a polymer. In any embodiment, the back plate 207 and the side bezel structure 206 may be integrally formed and may include the same material (e.g., a metal material such as aluminum). The binding members 250 and 260 may be formed of various materials and in various shapes. The binding members 250 and 260 may be formed as an integral type or a plurality of unit links which are capable of being moved with respect to each other by woven fabric, leather, rubber, urethane, metal, ceramic, or a combination of at least two of the materials.

According to an embodiment, the electronic device 200 may include at least one or more of a display 220 (as seen in 220 of FIG. 4), an audio module (205, 208), a sensor module 211, a key input device (202, 203, 204), and a connector hole 209. In any embodiment, the electronic device 200 may not include at least one (e.g., the key input device (202, 203, 204), the connector hole 209, or the sensor module 211) of the components or may further include any other component.

The display 220 may be exposed, for example, through a considerable portion of the front plate 201. A shape of the display 220 may be a shape corresponding to a shape of the front plate 201, and may have various shapes such as a circle, an oval, a polygon, or the like. The display 220 may be coupled to a touch sensing circuit, a pressure sensor capable of measuring the intensity (or pressure) of a touch, and/or a fingerprint sensor or may be disposed adjacent thereto.

The audio module (205, 208) may include the microphone hole and the speaker hole. A microphone for obtaining external sound may be disposed within the microphone hole; in any embodiment, a plurality of microphones may be disposed to detect a direction of sound. The speaker hole may be used as an external speaker and a call receiver. In any embodiment, the speaker hole and the microphone hole may be implemented with one hole, or a speaker (e.g., a piezo speaker) may be included without the speaker hole.

The sensor module 211 may generate an electrical signal or a data value that corresponds to an internal operation state of the electronic device 200 or corresponds to an external environment state. The sensor module 211 may include, for example, the biometric sensor module 211 (e.g., an HRM sensor) disposed on the second surface 210B of the housing 210. The electronic device 200 may further include a sensor module not illustrated, for example, at least one of a gesture sensor, a gyro sensor, a barometric pressure sensor, a magnetic sensor, an acceleration sensor, a grip sensor, a color sensor, an infrared (IR) sensor, a biometric sensor, a temperature sensor, a humidity sensor, or an illumination sensor.

The key input device (202, 203, 204) may include the wheel key 202 disposed on the first surface 210A of the housing 210 and capable of rotating in at least one direction, and/or a side key button (202, 203) disposed on the side surface 210C of the housing 210. A shape of the wheel key 202 may correspond to the shape of the front plate 201. In another embodiment, the electronic device 200 may not include all or part of the key input device (202, 203, 204) mentioned above, and the key input device (202, 203, 204) not included may be implemented on the display 220 in the form of a soft key or the like. The connector hole 209 may include other connector holes capable of accommodating a connector (e.g., a USB connector) for transmitting/receiving power and/or data with an external electronic device and accommodating a connector for transmitting/receiving an audio signal with the external electronic device. For example, the electronic device 200 may further include a connector cover (not illustrated) that covers at least a portion of the connector hole 209 and blocks the introduction of external foreign substances to the connector hole.

The binding members 250 and 260 may be removably bound to at least a partial area of the housing 210 by using locking members 251 and 261. The binding members 250 and 260 may include at least one or more of a fixing member 252, a fixing member fastening hole 253, a band guide member 254, and a band fixing ring 255.

The fixing member 252 may be configured to fix the housing 210 and the binding members 250 and 260 to a part of the body (e.g., a wrist, an ankle, or the like) of the user. The fixing member fastening hole 253 may fix the housing 210 and the binding members 250 and 260 to the part of the body of the user through the fixing member 252. The band guide member 254 may be configured to limit a movement range of the fixing member 252 when the fixing member 252 is fastened with the fixing member fastening hole 253, and thus, the binding members 250 and 260 may be bound to the part of the user's body so as to be in close contact therewith. In a state where the fixing member 252 is fastened to the fixing member fastening hole 253, the band fixing ring 255 may limit the movement range of the binding members 250 and 260.

Referring to FIG. 4, an electronic device 400 (e.g., the electronic device 200) may include a side bezel structure 410, a wheel key 420, the front plate 201, the display 220, a first antenna 450, a second antenna 455, a support member 460 (e.g., a bracket), a battery 470, a printed circuit board 480, a sealing member 490, a back plate 493, and binding members 495 and 497. At least one of the components of the electronic device 400 may be identical or similar to at least one of the components of the electronic device 200 of FIG. 2 or 3, and thus, additional description will be omitted to avoid redundancy. The support member 460 may be disposed within the electronic device 400, and the support member 460 may be connected with the side bezel structure 410 or may be integrally formed with the side bezel structure 410. For example, the support member 460 may be formed of a metal material and/or a nonmetal material (e.g., polymer). The display 220 may be coupled to one surface of the support member 460, and the printed circuit board 480 may be coupled to an opposite surface of the printed circuit board 480. A processor, a memory, and/or an interface may be mounted on the printed circuit board 480. The processor may include, for example, one or more of a central processing unit, an application processor, a graphic processing device (GPU), a sensor processor, or a communication processor.

The memory may include, for example, a volatile memory or a nonvolatile memory. The interface may include, for example, a high definition multimedia interface (HDMI), a universal serial bus (USB) interface, an SD card interface, and/or an audio interface. The interface may electrically or physically connect, for example, the electronic device 400 with an external electronic device and may include a USB connector, an SD card/MMC connector, or an audio connector.

The battery 470 that is a device for supplying a power to at least one component of the electronic device 400 may include, for example, a primary cell incapable of being recharged, a secondary cell rechargeable, or a fuel cell. At least a portion of the battery 470 may be disposed on substantially the same plane as the printed circuit board 480, for example. The battery 470 may be integrally disposed within the electronic device 400 or may be disposed to be removable from the electronic device 400.

The first antenna 450 may be interposed between the display 220 and the support member 460. The first antenna 450 may include, for example, a near field communication (NFC) antenna, an antenna for wireless charging, and/or a magnetic secure transmission (MST) antenna. For example, the first antenna 450 may perform short range communication with an external device or may wirelessly transmit/receive a power utilized to charge, and may send a magnetic-based signal including a short range communication signal or payment data. In another embodiment, an antenna structure may be formed by a part of the side bezel structure 410 and/or the support member 460, or by a combination thereof.

The second antenna 455 may be interposed between the circuit board 480 and the back plate 493. The second antenna 455 may include, for example, a near field communication (NFC) antenna, an antenna for wireless charging, and/or a magnetic secure transmission (MST) antenna. For example, the second antenna 455 may perform short range communication with an external device or may wirelessly transmit/receive a power utilized to charge, and may send a magnetic-based signal including a short range communication signal or payment data. In another embodiment, an antenna structure may be formed by a part of the side bezel structure 410 and/or the back plate 493, or by a combination thereof.

The sealing member 490 may be placed between the side bezel structure 410 and the back plate 493. The sealing member 490 may be configured to block moisture or external foreign substances from being introduced into a space surrounded by the side bezel structure 410 and the back plate 493.

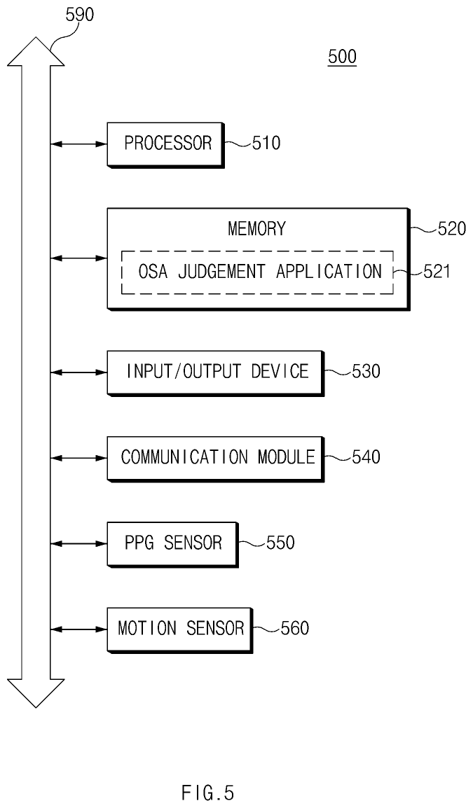

FIG. 5 is a block diagram illustrating an electronic device according to an embodiment of the disclosure.

According to an embodiment, an electronic device 500 (e.g., the electronic device 101 of FIG. 1 or the electronic device 200 of FIG. 2) may include a processor 510 (e.g., the processor 120), a memory 520 (e.g., the memory 130), a input/output device 530 (e.g., the input device 150, the sound output device 155, the display device 160), a communication module 540 (e.g., the communication module 190), a PPG sensor 550 (e.g., the sensor module 176), and a motion sensor 560 (e.g., the sensor module 176). The processor 510, the memory 520, the input/output device 530, the communication module 540, the PPG sensor 550, and the motion sensor 560 may exchange data with each other through a system bus 590. However, the configuration of the electronic device 500 is not limited thereto. According to certain embodiments, the electronic device 500 may not include at least one of the components described above or may further include at least any other component.

According to an embodiment, the processor 510 may perform operations or data processing associated with a control and/or communication of at least one another component (e.g., the memory 520, the input/output device 530, the communication module 540, the PPG sensor 550, and the motion sensor 560) of the electronic device 500. For example, the processor 510 may control a plurality of hardware or software components connected with the processor 510 by driving an operating system (e.g., the operating system 142) or an application program (e.g., an OSA screening application 521 (or an OSA judgement application 521)) and may perform various kinds of data processing and computation (or calculation).

According to an embodiment, the processor 510 may drive the obstructive sleep apnea (OSA) screening application 521. For example, the processor 510 may calculate biometric information (e.g., a heart rate or an oxygen saturation) of the user based on at least one information received from the PPG sensor 550 and may determine whether a user breathes, by using a calculation result. The processor 510 may determine a posture of the user based on at least one information received from the motion sensor 560. Based on basic biometric information (e.g., age, gender, height, weight, neck circumference, waist circumference, or hip circumference) of the user entered and information obtained from the PPG sensor 550 or the motion sensor 560, the processor 510 may analyze the obtained information to stepwise screen obstructive sleep apnea and may display an obstructive sleep apnea risk state of the user depending on a screening result. The processor 510 may provide the screening result through the input/output device 530 or may send the screening result to any other electronic device connected through the communication module 540.

According to certain embodiments, the processor 510 may send the basic biometric information of the user entered and at least one information received from the PPG sensor 550 or the motion sensor 560 to another electronic device (e.g., the electronic device 102, the electronic device 104, or the server 108) connected through the communication module 540, and the another electronic device may screen obstructive sleep apnea based on the at least one information received through the communication module 540.

According to an embodiment, the memory 520 may store a command or data associated with at least one another component (e.g., the processor 510, the input/output device 530, the communication module 540, the PPG sensor 550, and the motion sensor 560) of the electronic device 500. For example, the memory 520 may store the basic biometric information (e.g., age, gender, height, weight, neck circumference, waist circumference, or hip circumference) of the user entered, the information obtained from the PPG sensor 550 or the motion sensor 560, or the OSA screening application 521.

According to an embodiment, the input/output device 530 (e.g., a display or a speaker) may receive at least one information from the user or may provide at least one information to the user. In other words, the display (e.g., the display 220) may include a touch panel. The electronic device 500 may receive basic biometric information of the user through the display. The electronic device 500 may display at least one information about obstructive sleep apnea screening through the display or may output a sound corresponding to the at least one information through a speaker. According to certain embodiments, the input/output device 530 may further include a microphone, and may perform a user command based on sound information input through the microphone.

According to an embodiment, the communication module 540 may establish a direct (or wired) communication channel or a wireless communication channel between the electronic device 500 and another electronic device (e.g., the electronic device 102, the electronic device 104, or the server 108) and may perform communication through the established communication channel. For example, the communication module 540 may include the wireless communication module 192 (e.g., a cellular communication module, a short range wireless communication module, or a global navigation satellite system (GNSS) communication module) or the wired communication module 194 (e.g., a local area network (LAN) communication module or a power line communication module). The electronic device 500 may exchange at least one information about obstructive sleep apnea screening with another electronic device through the communication module 540.

According to an embodiment, the PPG sensor 550 may obtain at least one information associated with obstructive sleep apnea screening. For example, the PPG sensor 550 may include at least one light emitting unit and at least one light receiving unit. The light emitting unit may include an LED (e.g., an IR LED, a red LED, a green LED, or a blue LED, or the like) that emits a light. The light receiving unit may include at least one photodiode. The PPG sensor 550 may receive, through the light receiving unit, a light that is incident after a light output from the light emitting unit is reflected by a skin of the user. The PPG sensor 550 may transfer at least one information (e.g., a PPG sensor signal) associated with the light received through the light receiving unit. The processor 510 may perform measurement and identification on the following, based on the signal (or information) obtained through the PPG sensor 550: a heart rate of the user, an oxygen saturation of the user, or whether the user breathes. According to certain embodiments, the processor 510 may determine whether the electronic device 500 (e.g., a wrist-mounted wearable device) is mounted, through the PPG sensor 550.

According to certain embodiments, the PPG sensor 550 may measure a PPG sensor signal including a first parameter (e.g., a high-frequency component) and a second parameter (e.g., a low-frequency component), For example, the first parameter may refer to a high-frequency component having a frequency of about 1 Hz. The second parameter may refer to a low-frequency component having a frequency of about 0.2 Hz.

According to an embodiment, the motion sensor 560 may obtain at least one information associated with obstructive sleep apnea screening. For example, the motion sensor 560 may include an acceleration sensor and an angular velocity sensor. The motion sensor 560 may obtain at least one signal that is variable depending on a movement of the user. The processor 510 may determine an action and a state of the user (e.g., an action of lying down, a lying posture, an action of getting up, or a standing posture) based on the signal obtained by the motion sensor 560. According to certain embodiments, the processor 510 may determine an action and a state of the user based on the signals obtained by the motion sensor 560 and the PPG sensor 550.

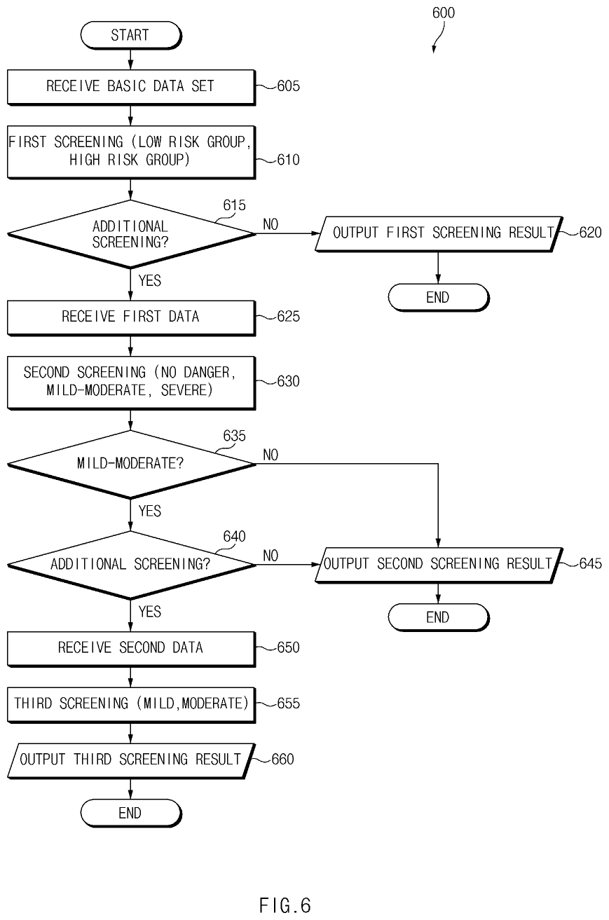

FIG. 6 is a flowchart illustrating an obstructive sleep apnea screening method 600 of an electronic device according to an embodiment of the disclosure. Table 1 below refers to a table in which risk degrees of obstructive sleep apnea are classified, according to an embodiment. The electronic device 500 may classify and guide risk degrees for obstructive sleep apnea of the user through the obstructive sleep apnea screening method 600, as shown in Table 1.

TABLE 1

| Stages | Classification of risk groups | | | |
|---|---|---|---|---|
| First screening | Low risk | | High risk | |
| Second screening | No danger | Mild-moderate | | Severe |
| Third screening | N/A | Mild | moderate | N/A |

Referring to FIGS. 5 and 6, the electronic device 500 may stepwise screen a risk state (e.g., no danger, a mild case, a moderate case, or a severe case) of obstructive sleep apnea based on obtained biometric information (e.g., a PPG sensor signal or a heart rate) of the user. For example, the processor 510 of the electronic device 500 may drive the OSA screening application 521 and may perform screening operations (e.g., a first screening operation, a second screening operation, or a third screening operation) for obstructive sleep apnea. The OSA screening application 521 may be executed depending on a user input or may be automatically activated at a specified time (e.g., a user sleep time). Alternatively, the processor 510 may output a test icon associated with a breath-holding test and an orthostatic test, which are associated with an obstructive sleep apnea risk degree test, on the display, may activate the OSA screening application 521 when selected by the user, and may perform at least one screen interface associated with the selected test and a sensor operation associated with a test progress.

According to an embodiment, in operation 605, the processor 510 of the electronic device 500 may receive a basic data set. For example, the basic data set may include information about a body of the user, such as, for example, basic biometric information including an age, gender, height, weight, neck circumference, waist circumference, or hip circumference of the user.

According to an embodiment, in operation 610, the processor 510 of the electronic device 500 may perform the first screening operation based on the basic data set. For example, the processor 510 may screen a state of the user through the first screening operation to sort the user into one of two states (e.g., a low risk group and a high risk group). The processor 510 may perform the first screening operation based on screening data (e.g., a data set indicating higher prevalence of obstructive sleep apnea in men, middle-aged and older, and obese people”) that is associated with obstructive sleep apnea. The screening data associated with obstructive sleep apnea may be in prestored in the memory 520.

According to an embodiment, in operation 615, the processor 510 of the electronic device 500 may receive a user input requesting execution or omission of an additional screening operation. For example, the processor 510 may receive an user input through the input/output device 530. When the user input requesting omission of an additional screening operation is received, in operation 620, the processor 510 may output a first screening result (e.g., a high risk group or a low risk group) through the input/output device 530. When the user input requests execution of an additional screening operation is received, the processor 510 may perform operation 625.

According to an embodiment, in operation 625, the processor 510 of the electronic device 500 may receive first data (e.g., a PPG sensor signal measured during sleep). For example, while the user sleeps, the processor 510 may receive the PPG sensor signal through the PPG sensor 550. The PPG sensor signal may include a high-frequency component associated with a heartrate (e.g., a “heartbeat frequency”) and a low-frequency component associated with a respiration rate (e.g., a “respiration frequency”).

According to an embodiment, in operation 630, the processor 510 of the electronic device 500 may perform the second screening operation based on the first data. For example, the processor 510 may screen a state of the user through the second screening operation as three states (e.g., no danger, a mild-moderate case, and a severe case). In normal, healthy breathing, the PPG sensor signal may detect both the high-frequency component and the low-frequency component and may have “great” frequency fluctuations, with “great” meaning higher than or equal to the predetermined fluctuation threshold, higher than another predefined threshold, or found within another predefined fluctuation range. In contrast, when a sleep apnea disorder is present, the PPG sensor signal may indicate the high-frequency component (e.g., to the exclusion of other components), to the exclusion of the low frequency component, and the high-frequency component may include “small” frequency fluctuations, “small” meaning below a predetermined fluctuation threshold, or within a predefined fluctuation range. The processor 510 may perform the second screening operation based on the frequency fluctuations of the PPG sensor signal.

According to certain embodiments, the processor 510 of the electronic device 500 may perform the second screening operation in consideration of the first screening result. For example, when the first screening result indicates a low risk group and the frequency fluctuations of the PPG sensor signal is “great,” the processor 510 may determine a second screening result as “no danger” categorization. When the first screening result indicates a high risk group and the frequency fluctuations of the PPG sensor signal is small, the processor 510 may determine the second screening result as a “severe” case. In the remaining cases (e.g., where the first screening result indicates a low risk group and the frequency fluctuations of the PPG sensor signal is small, or where the first screening result indicates a high risk group and the frequency fluctuations of the PPG sensor signal is “great”), the processor 510 may determine the second screening result as a “mild-moderate” case.

According to an embodiment, in operation 635, the processor 510 of the electronic device 500 may determine whether the state of the user is a mild-moderate state. For example, when the second screening result does not correspond to a mild-moderate case (e.g., when the second screening result corresponds to no danger or a severe case), the processor 510 may omit performance of an additional screening operation any longer; in operation 645, the processor 510 may output the second screening result (e.g., no danger or a severe case) through the input/output device 530. When the second screening result corresponds to a mild-moderate case, the processor 510 may perform operation 640.

According to an embodiment, in operation 640, the processor 510 of the electronic device 500 may receive a user input requesting either execution or omission of an additional screening operation. For example, the processor 510 may receive the user input through the input/output device 530. When the user input indicating to omit the additional screening operation is received, in operation 645, the processor 510 may output the second screening result (e.g., no danger or a severe case) through the input/output device 530. When the user input request execution of an additional screening operation is received, the processor 510 may perform operation 650.

According to an embodiment, in operation 650, the processor 510 of the electronic device 500 may receive second data (e.g., respiration state information, or movement information). For example, the processor 510 may display prompts through the input/output device 530 that guide the user to perform a breath-holding test, or an orthostatic test. Through the PPG sensor 550, the processor 510 may measure a heart rate of the user during the breath-holding test or the orthostatic test. Through the motion sensor 560, the processor 510 may measure information about a movement (e.g., an acceleration or an angular velocity) of the user during the breath-holding test or the orthostatic test.

According to an embodiment, in operation 655, the processor 510 of the electronic device 500 may perform a third screening operation based on the second data. For example, the processor 510 may screen a state of the user through the third screening operation to categorize the user into one of two states (e.g., a “mild” case and a “moderate” case), depending on the rests of the breath-holding test and/or the orthostatic test. That is, the sensor values (e.g., heart rate, motion) may be compared to pre-stored ranges or thresholds pre-associated with “mild” and “moderate” sleep apnea categorizations, to determine which category the user's sensor values sort into.

According to certain embodiments, the processor 510 of the electronic device 500 may determine a third screening result (e.g., a mild case or a moderate case) depending on a rate of change in a heart rate during the breath-holding test. For example, the breath-holding test may include inhaling for a specific time (e.g., about 2 seconds) and then holding breath for a specific time (e.g., about 15 seconds). When the heart rate changes rapidly during the breath-holding test, the processor 510 may determine the third screening result as a mild case. When the heart rate changes slowly during the breath-holding test, the processor 510 may determine the third screening result as a severe case.

According to certain embodiments, the processor 510 of the electronic device 500 may determine the third screening result (e.g., a mild case or a moderate case) depending on a rate of change in a heart rate during the orthostatic test. For example, the orthostatic test may include lying down for a specific time (e.g., about 5 minutes) and standing up and maintaining an upright posture for a specific time (e.g., about 2 minutes). When the rate of change in the heart rate is “great” during the orthostatic test (e.g., meaning falling within some heartrate threshold range predefined as “great,” or above a certain threshold of change in beats per minute), the processor 510 may determine the third screening result as a mild case. When the rate of change in the heart rate is “small” during the orthostatic test (e.g., meaning falling within some other heartrate threshold range predefined as “small,” or below the threshold of change in beats per minute), the processor 510 may determine the third screening result as a moderate case.

According to certain embodiments, the processor 510 of the electronic device 500 may determine the accuracy of action and posture of the user based on a portion (e.g., movement information) of the second data during the breath-holding test and the orthostatic test. The processor 510 may generate the third screening result in consideration of the breath-holding test result and the orthostatic test result. For example, the processor 510 may determine the accuracy of the breath-holding test result by using the orthostatic test result.

According to an embodiment, in operation 660, the processor 510 of the electronic device 500 may output the third screening result (e.g., a mild case or a moderate case) through the input/output device 530.

As described above, the electronic device 500 may stepwise perform screening operations (e.g., the first screening operation, the second screening operation, and the third screening operation) and may finally classify an obstructive sleep apnea risk state into four cases (e.g., no danger, a mild case, a moderate case, and a severe case).

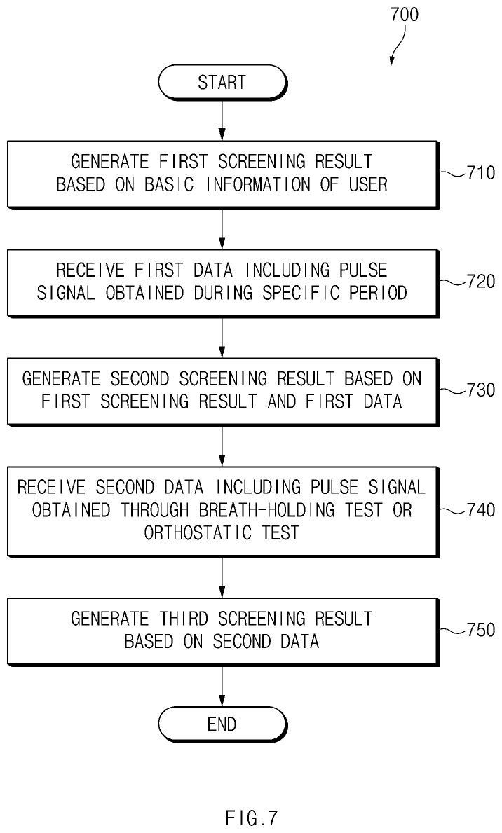

FIG. 7 is a flowchart illustrating an obstructive sleep apnea screening method 700 of an electronic device according to certain embodiments of the disclosure.

Referring to FIGS. 5 and 7, the electronic device 500 may stepwise screen a risk state (e.g., seventies of no danger, a mild case, a moderate case, or a severe case) (c.f., refer to Table 1) of obstructive sleep apnea based on obtained biometric information (e.g., a PPG sensor signal or a heart rate) of the user. For example, the processor 510 of the electronic device 500 may drive the OSA screening application 521 to perform stepwise screening operations (e.g., the first screening operation, the second screening operation, or the third screening operation) for obstructive sleep apnea.

According to an embodiment, in operation 710, the processor 510 of the electronic device 500 may generate a first screening result (e.g., sorting the user into a low risk group or a high risk group) based on basic biometric information (e.g., age, gender, height, weight, neck circumference, waist circumference, or hip circumference) of the user. For example, the processor 510 may receive the basic biometric information of the user through the input/output device 530. The processor 510 may perform the first screening operation based on the basic biometric information of the user. The memory 520 may store risk criterion information (e.g., information such as “the prevalence of obstructive sleep apnea is higher in men, middle-aged and older, and obese people”). The processor 510 may compare the basic biometric information of the user and the risk criterion information to generate the first screening result (e.g., if the user is a male, middle-aged and obese, he may be sorted into the high risk group).

According to an embodiment, in operation 720, the processor 510 of the electronic device 500 may receive first data (e.g., a heart rate) including a pulse signal (e.g., a PPG sensor signal) obtained during over a specified period of time (e.g., a full night's sleep). For example, as the user sleeps, the processor 510 may monitor the user and receive the PPG sensor signal (or a heart rate) through the PPG sensor 550 which may be operatively coupled to the user. The PPG sensor signal may include a first parameter (e.g., a high-frequency component associated with a heartbeat frequency) and a second parameter (e.g., a low-frequency component associated with a respiration frequency).

According to an embodiment, in operation 730, the processor 510 of the electronic device 500 may generate a second screening result (e.g., no danger, a mild-moderate case, and a severe case) based on the first screening result (e.g., a high risk group or a low risk group) and the first data. For example, the processor 510 may perform the second screening operation based on frequency fluctuations of the PPG sensor signal (or heart rate). In normal breathing, the PPG sensor signal (or heart rate) may include both the high-frequency component and the low-frequency component and may have “great” frequency fluctuations. In contrast, in sleep apnea, the PPG sensor signal (or heart rate) may include the high-frequency component (e.g., to the exclusion of other components) and may have “small” frequency fluctuations.

According to certain embodiments, the processor 510 of the electronic device 500 may perform the second screening operation in consideration of the first screening result. For example, when the first screening result indicates a low risk group and the frequency fluctuations of the PPG sensor signal is great, the processor 510 may determine a second screening result as no danger. When the first screening result indicates a high risk group and the frequency fluctuations of the PPG sensor signal is small, the processor 510 may determine the second screening result as a severe case. In the remaining cases (e.g., the case where the first screening result indicates a low risk group and the frequency fluctuations of the PPG sensor signal is small or the case where the first screening result indicates a high risk group and the frequency fluctuations of the PPG sensor signal is great), the processor 510 may determine the second screening result as a mild-moderate case.

According to an embodiment, in operation 740, the processor 510 of the electronic device 500 may receive second data (e.g., heart rate, acceleration, or angular velocity information) including a pulse signal (e.g., a PPG sensor signal or a motion sensor signal) obtained through a breath-holding test or the orthostatic test. For example, the processor 510 may prompt through the input/output device 530, the user to perform a breath-holding test or an orthostatic test (e.g., in which on screen display information may guide the user through execution of the test). Through the PPG sensor 550 or the motion sensor 560, the processor 510 may measure a heart rate of the user during the breath-holding test or the orthostatic test.

According to an embodiment, in operation 750, the processor 510 of the electronic device 500 may generate a third screening result (e.g., a mild case or a moderate case) based on the second data. For example, through the third screening operation, the processor 510 may subdivide the mild-moderate cases into further sub-classifications of a “mild” case and a “moderate” case).

According to certain embodiments, the processor 510 of the electronic device 500 may determine a third screening result (e.g., a mild case or a moderate case) depending on a rate of change in a heart rate during the breath-holding test. For example, the breath-holding test may include inhaling for a specific time (e.g., about 2 seconds) and then holding breath for a specific time (e.g., about 15 seconds). When the heart rate changes rapidly during the breath-holding test, the processor 510 may determine the third screening result as a mild case. When the heart rate changes slowly during the breath-holding test, the processor 510 may determine the third screening result as a moderate case.

According to certain embodiments, the processor 510 of the electronic device 500 may determine the third screening result (e.g., a mild case or a moderate case) depending on a rate of change in a heart rate during the orthostatic test. For example, the orthostatic test may include lying down for a specific time (e.g., about 5 minutes) and standing up and maintaining an upright posture for a specific time (e.g., about 2 minutes). When the rate of change in the heart rate is great during the orthostatic test, the processor 510 may determine the third screening result as a mild case. When the rate of change in the heart rate is small during the orthostatic test, the processor 510 may determine the third screening result as a moderate case.

According to certain embodiments, the processor 510 of the electronic device 500 may determine the accuracy of action and posture of the user based on a portion (e.g., movement information) of the second data during the breath-holding test and the orthostatic test. The processor 510 may generate the third screening result in consideration of the breath-holding test result and the orthostatic test result. For example, the processor 510 may determine the accuracy of the breath-holding test result by using the orthostatic test result.

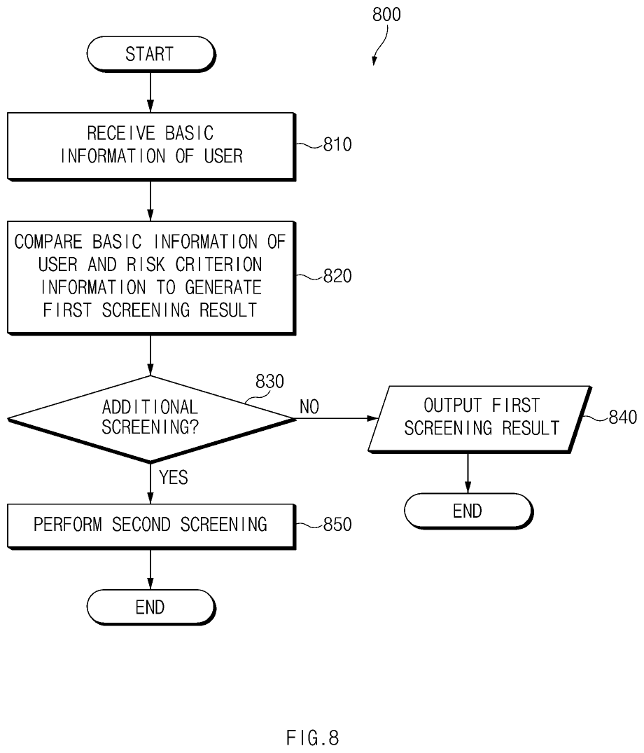

FIG. 8 is a flowchart illustrating an example 800 of a method in which an electronic device performs a first screening operation in FIG. 7.

Referring to FIGS. 5 and 8, the electronic device 500 may perform the first screening operation based on basic biometric information (e.g., age, gender, height, weight, neck circumference, waist circumference, or hip circumference) of the user.

According to an embodiment, in operation 810, the processor 510 of the electronic device 500 may receive basic biometric information of the user. For example, the processor 510 may receive the basic biometric information of the user through the input/output device 530. Alternatively, the basic biometric information of the user may be in prestored in the memory 520.

According to an embodiment, in operation 820, the processor 510 of the electronic device 500 may compare the basic biometric information of the user and the risk criterion information, and generate a first screening result (e.g., a low risk group or a high risk group) (refer to Table 1). In this regard, the memory 520 may prestore the risk criterion information (e.g., information such as “the prevalence of obstructive sleep apnea is higher in men, middle-aged and older, and obese people”). This information may then be used, by way of comparison against the basic biometric information, to sort the user into one of a number of severity categorizations for sleep apnea.

According to an embodiment, in operation 830, the processor 510 of the electronic device 500 may receive a user input associated with whether to perform an additional screening operation. For example, the processor 510 may guide participation for more detailed determination through the input/output device 530. When the user input indicating to perform no additional screening operation is received, in operation 840, the processor 510 may output the first screening result (e.g., a high risk group or a low risk group) through the input/output device 530. When the user input indicating to perform an additional screening operation is received, in operation 850, the processor 510 may perform the second screening operation.

According to certain embodiments, in operation 840, the processor 510 of the electronic device 500 may provide an appropriate management method of obstructive sleep apnea through the input/output device 530 based on the first screening result (e.g., a high risk group or a low risk group).

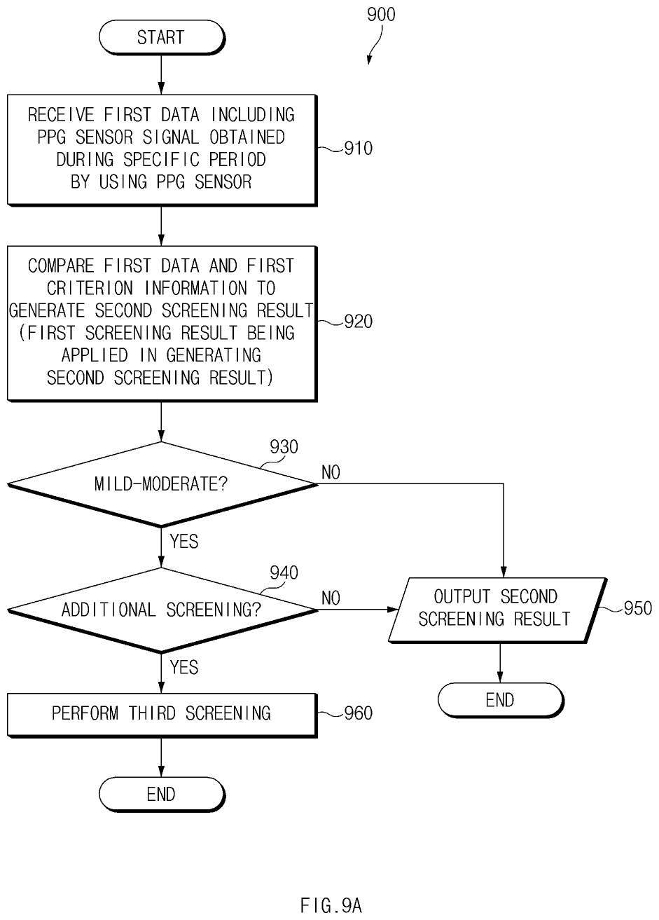

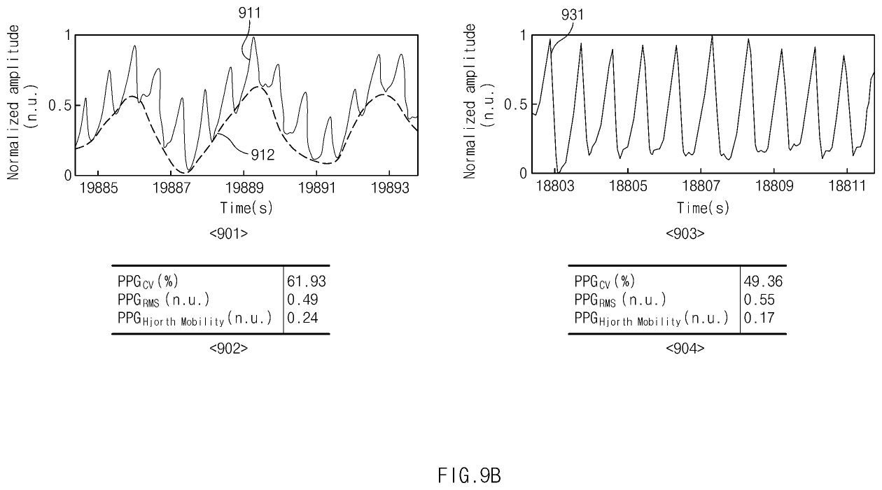

FIG. 9A is a flowchart illustrating an example 900 of a method in which an electronic device performs a second screening operation in FIG. 7. FIG. 9B is a graph illustrating a normal state and an obstructive sleep apnea risk state according to an embodiment.

Referring to FIGS. 5 and 9A, the processor 510 of the electronic device 500 may perform the second screening operation based on first data including a pulse signal obtained during a specific period.

According to an embodiment, in operation 910, the processor 510 of the electronic device 500 may receive the first data (e.g., a heart rate), which include a PPG sensor signal obtained over a specified period (e.g., one night's sleep), through the PPG sensor 550. For example, while the user sleeps, the processor 510 may receive the PPG sensor signal (or a heart rate) through the PPG sensor 550. The PPG sensor signal may include a first parameter (e.g., a high-frequency component associated with a heartbeat frequency) and a second parameter (e.g., a low-frequency component associated with a respiration frequency).

According to an embodiment, in operation 920, the processor 510 of the electronic device 500 may compare the first data and first criterion information, and generate a second screening result (e.g., no danger, a mild-moderate case, or a severe case) (c.f., refer to Table 1). For example, the processor 510 may perform the second screening operation based on the frequency fluctuations of the PPG sensor signal (or heart rate). In this regard, the memory 520 may prestore the first criterion information.

According to certain embodiments, the first criterion information may include at least part of frequency fluctuation graphs 901 and 903 or frequency fluctuation tables 902 and 904, as illustrated in FIG. 9B. The graph 901 and the table 902 show the frequency fluctuations of the PPG sensor signal (or heart rate) in normal breathing. The graph 903 and the table 904 show the frequency fluctuations of the PPG sensor signal (or heart rate) in sleep apnea. For example, in the table 902 and the table 904, a value of “PPG_{Hjorth Mobility}” may be a parameter showing the frequency fluctuations. “Hjorth Mobility” may be calculated by Equation 1 below. y(t) represents a measured signal (e.g., a PPG sensor signal).

$$Hjorth Mobility = \sqrt{var ( \frac{dy ( t )}{dt} ) / var ( y ( t ) )} \qquad [ Equation 1 ]$$

In normal breathing, the PPG sensor signal (or heart rate) may include both a high-frequency component 911 and a low-frequency component 912 and may have great frequency fluctuations (e.g., refer to a “PPG_{Hjorth Mobility}” value in the table 902; as the “PPG_{Hjorth Mobility}” value becomes greater, the frequency fluctuations increase). In sleep apnea, the PPG sensor signal (or heart rate) may include a high-frequency component 931 (e.g., to the exclusion of other components) and may have small frequency fluctuations (e.g., refer to a “PPG_{Hjorth Mobility}” value in the table 904; as the “PPG_{Hjorth Mobility}” value becomes smaller, the frequency fluctuations decrease). The second screening operation utilizes the difficulty in measuring the second parameter (e.g., a low-frequency component or 912) of the PPG sensor signal due to a decrease in the amplitude of respiration airflow, which occurs repeatedly during sleep in people with severe obstructive sleep apnea.

According to certain embodiments, the processor 510 of the electronic device 500 may perform the second screening operation in consideration of the first screening result. For example, when the first screening result indicates a low risk group and the frequency fluctuations of the PPG sensor signal is great (e.g., 901 and 902), the processor 510 may determine a second screening result as no danger. When the first screening result indicates a high risk group and the frequency fluctuations of the PPG sensor signal is small (e.g., 903 and 904), the processor 510 may determine the second screening result as a severe case. In the remaining cases (e.g., the case where the first screening result indicates a low risk group and the frequency fluctuations of the PPG sensor signal is small or the case where the first screening result indicates a high risk group and the frequency fluctuations of the PPG sensor signal is great), the processor 510 may determine the second screening result as a mild-moderate case.

According to an embodiment, in operation 930, the processor 510 of the electronic device 500 may determine whether the state of the user is a mild-moderate state. For example, when the second screening result does not correspond to a mild-moderate case (e.g., when the second screening result corresponds to no danger or a severe case), the processor 510 may omit performance of an additional screening operation; in operation 950, the processor 510 may output the second screening result (e.g., no danger or a severe case) through the input/output device 530. When the second screening result corresponds to a mild-moderate case, the processor 510 may perform operation 940.

According to an embodiment, in operation 940, the processor 510 of the electronic device 500 may receive a user input associated with whether to perform an additional screening operation. For example, the processor 510 may guide participation for more detailed determination (e.g., subdivision into a mild-moderate case) through the input/output device 530. When the user input indicating to perform no additional screening operation is received, in operation 950, the processor 510 may output the second screening result (e.g., no danger or a severe case) through the input/output device 530. When the user input requesting execution of an additional screening operation is received, in operation 960, the processor 510 may perform the third screening operation.

According to certain embodiments, in operation 950, the processor 510 of the electronic device 500 may provide an appropriate management method of obstructive sleep apnea through the input/output device 530 based on the second screening result (e.g., no danger, a mild-moderate case, or a severe case).

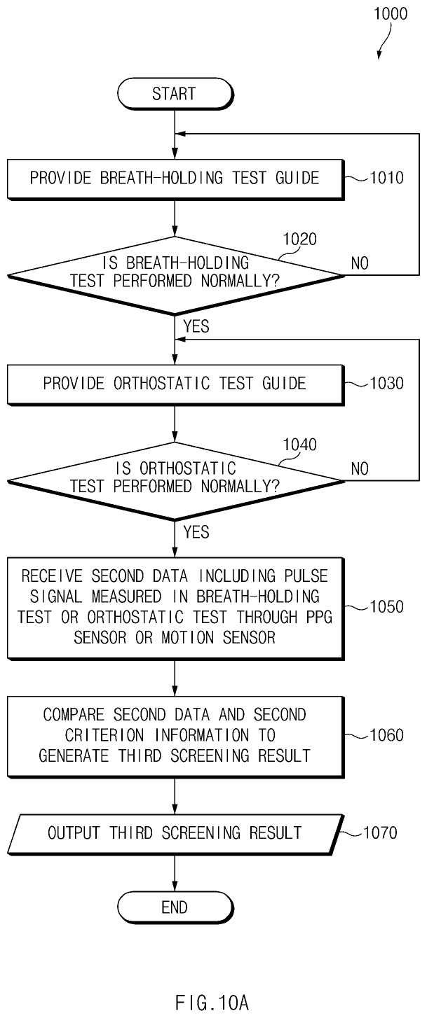

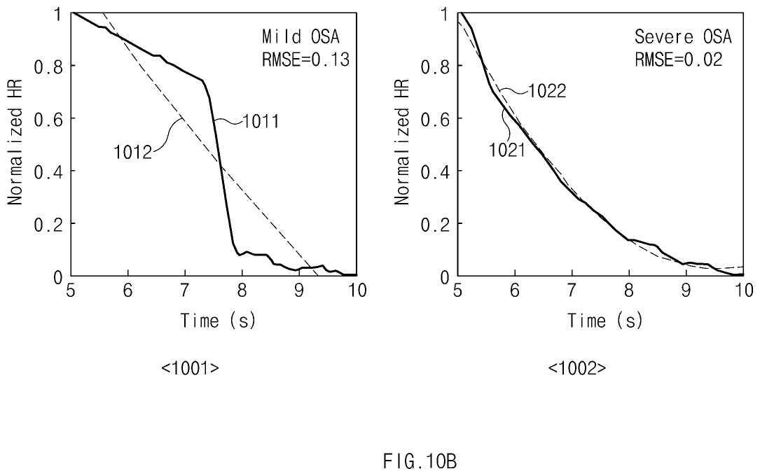

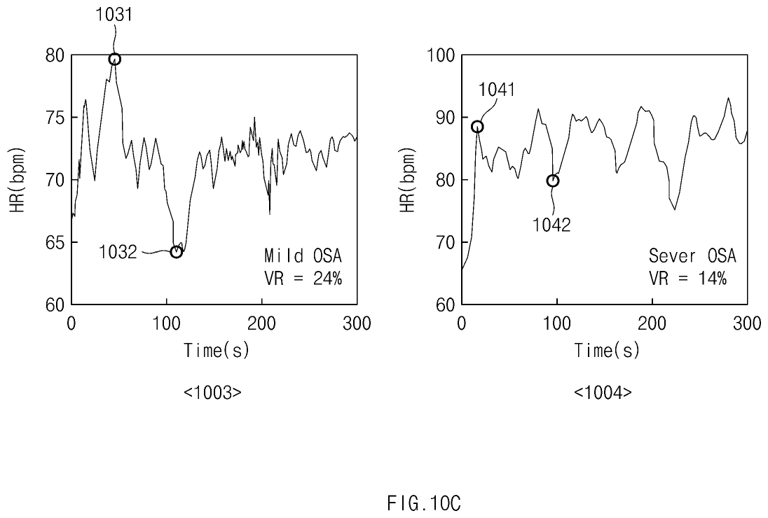

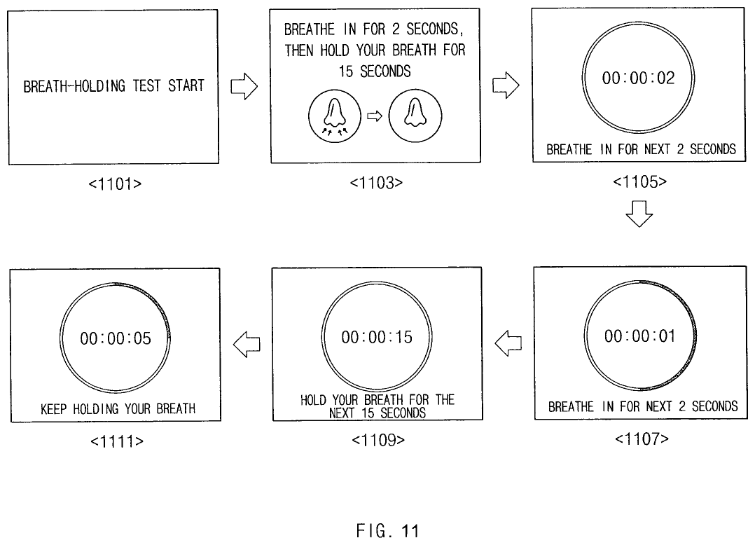

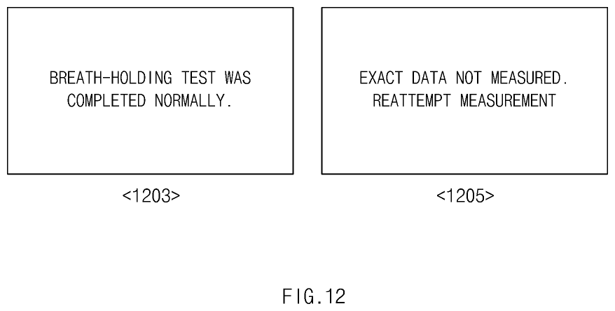

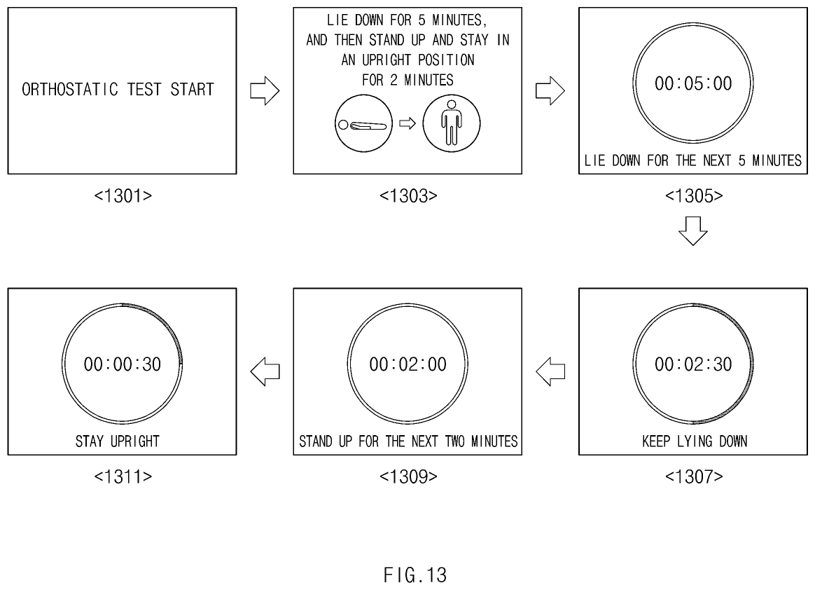

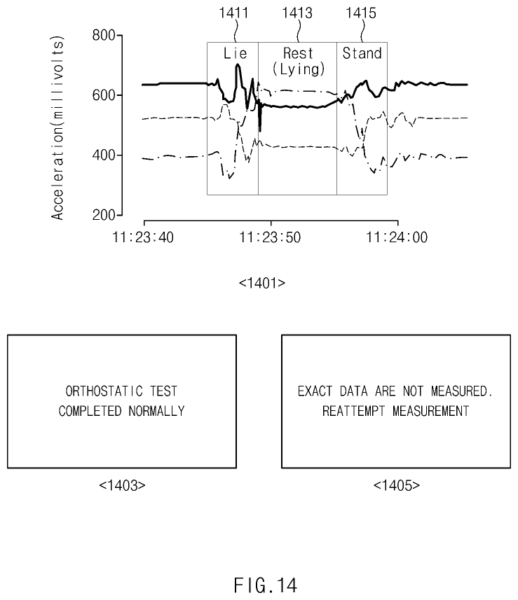

FIG. 10A is a flowchart illustrating an example 1000 of a method in which an electronic device performs a third screening operation in FIG. 7. FIG. 10B is a graph for determining a result of a breath-holding test according to an embodiment. FIG. 10C is a graph for determining a result of an orthostatic test according to an embodiment. FIG. 11 is a view illustrating a user interface in a breath-holding test of an electronic device according to an embodiment. FIG. 12 is a view illustrating a user interface associated with reexamination in a breath-holding test of an electronic device according to an embodiment. FIG. 13 is a view illustrating a user interface in an orthostatic test of an electronic device according to an embodiment. FIG. 14 is a view illustrating a user interface associated with reexamination in an orthostatic test of an electronic device according to an embodiment.

Referring to FIGS. 5 and 10A, the electronic device 500 may perform the third screening operation based on second data (e.g., respiration state information or movement information) including a pulse signal (e.g., a PPG sensor signal) obtained during a specific period.

According to an embodiment, in operation 1010, the processor 510 of the electronic device 500 may provide a visual guidance, through a display, prompting a user to execute a breath-holding test input/output device 530. For example, as illustrated in FIG. 11, the processor 510 may display guide screens 1101 to 1111 for the breath-holding test through the input/output device 530. The processor 510 may measure a PPG sensor signal (or a heart rate) through the PPG sensor 550 while at least part of the screens 1105 to 1111 is displayed. The breath-holding test may include inhaling for a specific time (e.g., about 2 seconds) and then holding breath for a specific time (e.g., about 15 seconds). Conditions associated with the breath-holding test may vary depending on user information, a region, generation, and the like.

According to an embodiment, in operation 1020, the processor 510 of the electronic device 500 may determine whether the breath-holding test was satisfactorily performed. For example, the processor 510 may determine whether the breath-holding test is performed normally without deviation into error, by using the graphs 901 and 903 of FIG. 9B. When the user is wearing the electronic device 500, and repeats an inhalation action for a specific time, and then holds the breath for a specific time, a resulting signal of the PPG sensor included in the electronic device 500 may be compared against those of the graphs 901 and 903 of FIG. 9B. When a pattern of the measured PPG sensor signal is different from that of the graphs 901 and 903 of FIG. 9B (e.g., by more than a preset difference threshold), the processor 510 may determine that the breath-holding test was performed abnormally. When the breath-holding test is performed abnormally, the processor 510 may prompt repeat performance of the holding test, by reverting to operation 1010. When the breath-holding test is performed abnormally, the processor 510 may display screen 1205 of FIG. 12 through the input/output device 530. When the breath-holding test is performed normally, the processor 510 may perform operation 1030. When the breath-holding test is performed normally, the processor 510 may display screen 1203 of FIG. 12 through the input/output device 530.

When the breath-holding test is performed satisfactorily and normally, then according to an embodiment, in operation 1030, the processor 510 of the electronic device 500 may provide an orthostatic test guide through the input/output device 530. For example, as illustrated in FIG. 13, the processor 510 may display at least part of screens 1301 to 1311 through the input/output device 530. The processor 510 may measure a PPG sensor signal (or a heart rate) through the PPG sensor 550 while the screens 1305 to 1311 are displayed. The orthostatic test may include lying down for a specific time (e.g., about 5 minutes), then standing up and maintaining an upright posture for a specific time (e.g., about 2 minutes). Conditions associated with the orthostatic test may vary depending on user information, a region, generation, and the like, which may be accounted for in the software.

According to an embodiment, in operation 1040, the processor 510 of the electronic device 500 may determine whether the orthostatic test was performed normally. For example, the processor 510 may determine whether the orthostatic test is normally performed, by using the motion sensor 560. The processor 510 may measure an acceleration and an angular velocity corresponding to a change in posture of the user by using the motion sensor 560. When the user wearing the electronic device 500 maintains the upright posture for the specific time after lying down for the specific time and standing up, the measured acceleration and angular velocity may be measured and should be similar to that in a graph 1401 of FIG. 14 (e.g., within a preset threshold of variation). When a pattern of the measured acceleration and angular velocity is different from patterns 1411, 1413 (e.g., greater than the preset variation threshold), and 1415 of the graph 1401 of FIG. 14, the processor 510 may identify that the orthostatic test has been performed abnormally. When the orthostatic test is performed abnormally, the processor 510 may prompt a repeat of the orthostatic test by reverting to operation 1030. When the orthostatic test is performed abnormally, the processor 510 may display screen 1405 of FIG. 14 through the input/output device 530. When the orthostatic test is performed normally, the processor 510 may continue on to operation 1050. When the orthostatic test is performed normally, the processor 510 may display screen 1403 of FIG. 14 through the input/output device 530.

According to an embodiment, in operation 1050, the processor 510 of the electronic device 500 may receive second data (e.g., heart rate, acceleration, or angular velocity information) including a pulse signal (e.g., a PPG sensor signal or a motion sensor signal) measured in the breath-holding test or the orthostatic test through the PPG sensor 550 or the motion sensor 560.

According to an embodiment, in operation 1060, the processor 510 of the electronic device 500 may compare the second data and second criterion information to generate a third screening result (e.g., a mild case or a moderate case). For example, the processor 510 may perform the third screening operation based on a change pattern of the heart rate or a rate of change in the heart rate. In this regard, the memory 520 may prestore the second criterion information.

According to certain embodiments, the second criterion information may include graphs 1001 and 1002 of FIG. 10B associated with a change pattern of the heart rate or graphs 1003 and 1004 of FIG. 10C associated with a rate of change in the heart rate. The graphs 1001 and 1003 show a change pattern of the heart rate or a rate of change in the heart rate in normal breathing. For example, a pattern (e.g., the graphs 1001 and 1003) in which a heart rate changes greatly may appear in normal breathing. The graphs 1002 and 1004 show a change pattern of the heart rate or a rate of change in the heart rate in sleep apnea. For example, a pattern (e.g., the graphs 1002 and 1004) in which a heart rate hardly changes may appear in sleep apnea. The processor 510 of the electronic device 500 may compare the second data with the graphs 1001 to 1004 to generate a third screening result (e.g., by determining whether the second data produces lines and curvatures similar to any of the graphs 1001 to 1004, within a predefined threshold variation thereof).

According to certain embodiments, the processor 510 of the electronic device 500 may determine the third screening result (e.g., a mild case or a moderate case) depending on a change pattern of a heart rate during the breath-holding test. For example, when the heart rate changes rapidly during the breath-holding test, the processor 510 may determine the third screening result as a mild case. The reason is that, in people who do not have sleep apnea, the heart rate changes rapidly due to mechanism of protection against less oxygen inflow into the body. When the heart rate changes slowly during the breath-holding test, the processor 510 may determine the third screening result as a moderate case. The reason is that, in people with a severe sleep apnea symptom, the heart rate changes slowly due to damage to the autonomic nervous system. To quantify this difference, the processor 510 may compare nonlinear regression lines 1012 and 1022 and heart rates 1011 and 1021 to determine the third screening result.

For example, the graph 1001 shows a change pattern of the heart rate 1011 of a person that does not have a sleep apnea symptom. As illustrated in the graph 1001, during normal breathing, the heart rate 1011 may change with a sharp difference from the nonlinear regression line 1012. The graph 1002 shows a change pattern of the heart rate 1021 of a person with a severe apnea symptom. As illustrated in the graph 1002, during sleep apnea, the heart rate 1021 may change almost without a difference from the nonlinear regression line 1022. A root mean square error of the nonlinear regression line 1012 during normal breathing (e.g., RMSE=about 0.13) may be measured to be greater than a root mean square error of the nonlinear regression line 1022 during sleep apnea (e.g., RMSE=about 0.02). Accordingly, the processor 510 of the electronic device 500 may check a root mean square error of a nonlinear regression line to determine the third screening result.

According to certain embodiments, the processor 510 of the electronic device 500 may determine the third screening result (e.g., a mild case or a moderate case) depending on a rate of change in a heart rate during the orthostatic test. For example, when the rate of change in the heart rate is great during the orthostatic test (e.g., in the case of 1031 and 1032 of FIG. 10C), the processor 510 may determine the third screening result as a mild case. When the rate of change in the heart rate is small during the orthostatic test (e.g., in the case of 1041 and 1042 of FIG. 10C), the processor 510 may determine the third screening result as a moderate case.

According to certain embodiments, the processor 510 of the electronic device 500 may determine the accuracy of action and posture of the user based on a portion (e.g., movement information) of the second data during the breath-holding test and the orthostatic test. The processor 510 may generate the third screening result in consideration of the breath-holding test result and the orthostatic test result. For example, the processor 510 may determine the accuracy of the breath-holding test result by using the orthostatic test result.

According to an embodiment, in operation 1070, the processor 510 of the electronic device 500 may output the third screening result (e.g., a mild case or a moderate case) through the input/output device 530.

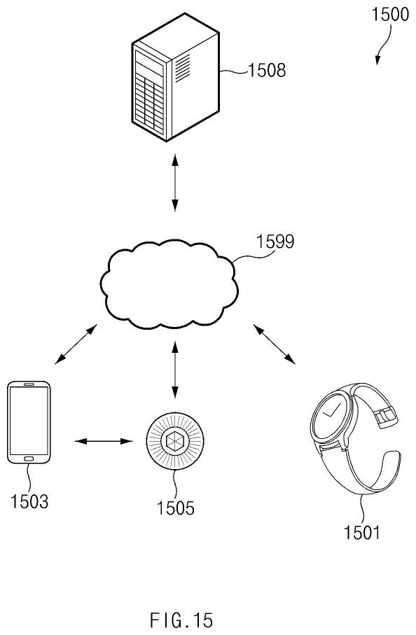

FIG. 15 is a diagram illustrating an electronic device in a network environment 1500 according to certain embodiments of the disclosure.

According to an embodiment, a first electronic device 1501 (e.g., the electronic device 500 or the electronic device 200) may communicate with a second electronic device 1503 (e.g., a smartphone or a tablet), a third electronic device 1505 (e.g., an Internet of things device), and a server 1508 over a network 1599. The first electronic device 1501 may include a sensor module (e.g., the PPG sensor 550 or the motion sensor 560) and a communication module (e.g., the communication module 540) without a display and a speaker. The first electronic device 1501 may send data for an obstructive sleep apnea screening operation (e.g., the first to third screening operations) to the second electronic device 1503, the third electronic device 1505, or the server 1508 over the network 1599. When receiving the data, the second electronic device 1503, the third electronic device 1505, or the server 1508 may determine a device including a user interface (e.g., a display or a speaker). The first electronic device 1501 may perform an obstructive sleep apnea screening operation while exchanging an output and a response with the determined device for each step.

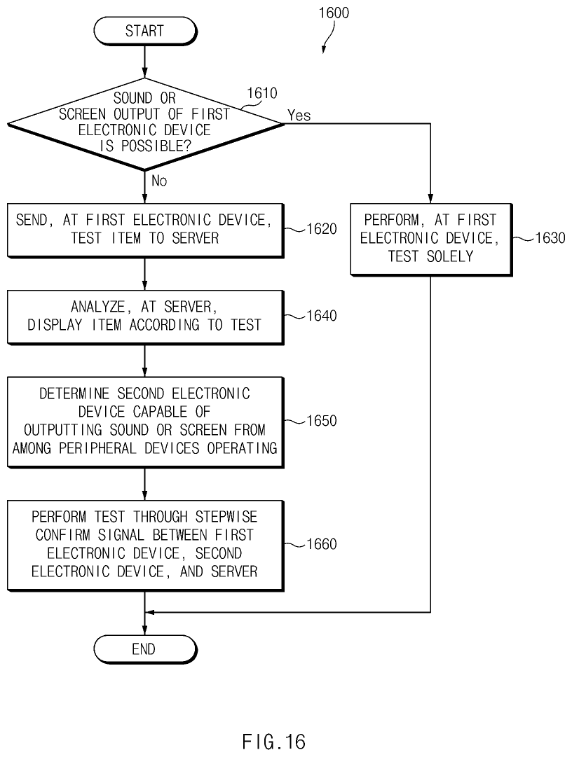

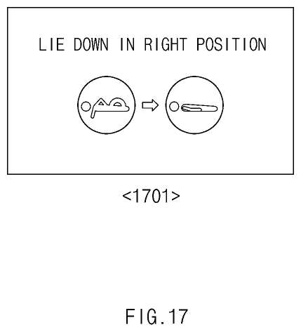

FIG. 16 is a flowchart illustrating an obstructive sleep apnea screening method 1600 of an electronic device in the network environment 1500 according to certain embodiments of the disclosure. FIG. 17 is a view illustrating a user interface associated with posture adjustment in an orthostatic test of an electronic device according to an embodiment.

According to an embodiment, in operation 1610, a first electronic device (e.g., the first electronic device 1501) may determine whether it is possible to output a sound or a screen. For example, when it is determined that it is possible to output a sound or a screen, in operation 1630, the first electronic device may perform an obstructive sleep apnea screening test “solely,” meaning without interfacing with an external device. When it is determined that it is impossible to output a sound or a screen, the first electronic device may omit performance of the OSA screen test solely, and proceed instead to operation 1620.

According to an embodiment, in operation 1620, the first electronic device may send the OSA screening test item to a server (e.g., the server 1508). For example, the first electronic device may send data items (e.g., basic biometric information of the user, measurement of a PPG sensor signal during sleep, a breath-holding test, or an orthostatic test) for the first, second and third screening operations to the server over a network (e.g., the network 1599).

According to an embodiment, in operation 1640, the server may analyze a display item according to a test. For example, the server may classify an item, which requires a screen display or a sound output, from among the items for the first to third screening operations thus received.

According to an embodiment, in operation 1650, the server may select a second electronic device, which is capable of outputting a sound or a screen, from among a plurality of peripheral devices (e.g., the second electronic device 1503 or the third electronic device 1505) to which it is connected over the network.

According to an embodiment, in operation 1660, the first electronic device, the second electronic device, and the server may perform the OSA screening test through a stepwise confirmation signal. For example, depending on a signal of the first electronic device, the second electronic device may output a sound or screen utilized for the first to third screening operations. Depending on a confirm signal of the second electronic device, the first electronic device may perform the first to third screening operations (e.g., measurement of a PPG sensor signal, a breath-holding test, or an orthostatic test). The confirm signal between the first electronic device and the second electronic device may be transferred through the server.

According to certain embodiments, in operation 1650, the server may select a second electronic device, capable of photographing an image, from among the plurality of peripheral devices. For example, in the orthostatic test, the second electronic device may analyze an action and a posture of the user through the image. The second electronic device may send information about the action and posture of the user to the first electronic device. When the action and posture of the user is incorrect, the first electronic device may provide a posture adjustment guide (e.g., screen 1701 of FIG. 17) through the second electronic device.

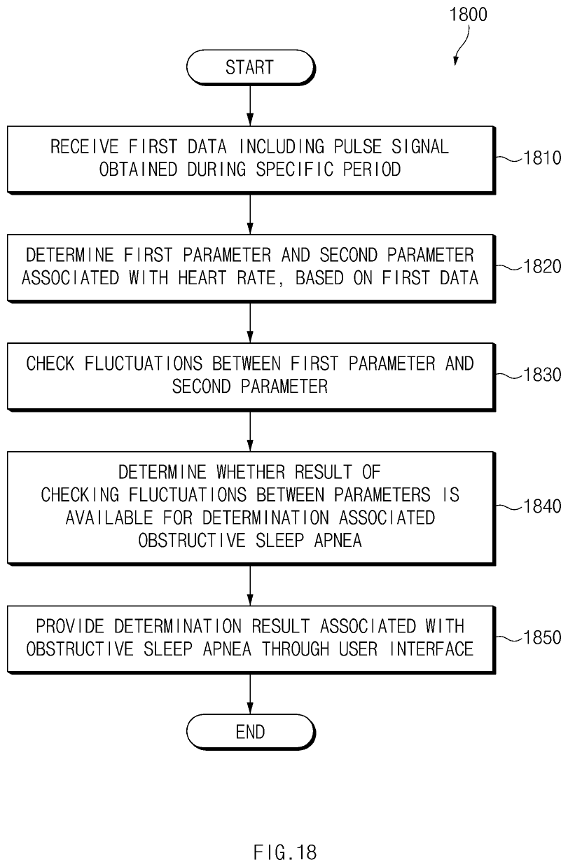

FIG. 18 is a flowchart illustrating a method 1800 in which an electronic device provides a result of determining obstructive sleep apnea, according to certain embodiments of the disclosure.

Referring to FIGS. 5 and 18, the electronic device 500 may stepwise screen a risk state (e.g., no danger, a mild case, a moderate case, or a severe case) (refer to Table 1) of obstructive sleep apnea based on obtained biometric information (e.g., a PPG sensor signal or a heart rate) of the user. For example, the processor 510 of the electronic device 500 may drive the OSA screening application 521 to perform stepwise screening operations (e.g., the first screening operation, the second screening operation, or the third screening operation) for obstructive sleep apnea.

According to an embodiment, in operation 1810, the processor 510 of the electronic device 500 may receive first data (e.g., a heart rate) including a pulse signal (e.g., a PPG sensor signal) obtained during a specific period (e.g., one night's sleep). For example, while the user sleeps, the processor 510 may receive the PPG sensor signal (or heart rate) through the PPG sensor 550.

According to an embodiment, in operation 1820, the processor 510 of the electronic device 500 may determine a first parameter and a second parameter associated with a heart rate, based on the first data. For example, the PPG sensor signal may include the first parameter (e.g., a high-frequency component associated with a heartbeat frequency) and the second parameter (e.g., a low-frequency component associated with a respiration frequency). The processor 510 may analyze the first data to extract the first parameter corresponding to the high-frequency component and the second parameter corresponding to the low-frequency component.

According to an embodiment, in operation 1830, the processor 510 of the electronic device 500 may analyze fluctuations found between the first parameter and the second parameter. For example, referring to FIG. 9B, the processor 510 may extract the first parameter (e.g., 911 or 931) and the second parameter (e.g., 912) from the frequency fluctuation graphs 901 and 903. In normal breathing, the processor 510 may extract the first parameter (e.g., 911) and the second parameter (e.g., 912) as in the graph 901. In sleep apnea, the processor 510 may extract the first parameter (e.g., 931) as in the graph 903, but may be unable to extract the second parameter, or may extract the second parameter having a very low frequency. The processor 510 may obtain values of the frequency fluctuations tables 902 and 903 of FIG. 9B by using the first parameter and the second parameter. For example, in the tables 902 and 904, a value of “PPG_{Hjorth Mobility}” shows fluctuations (e.g., fluctuations of a heart rate) between the first parameter and the second parameter. As a value of the parameter PPG_{Hjorth Mobility} becomes greater, the fluctuations between the first parameter and the second parameter may increase; as the fluctuations between the first parameter and the second parameter decrease (e.g., if the value is below a preset threshold), the processor 510 may determine an obstructive sleep apnea state as severe.

According to an embodiment, in operation 1840, the processor 510 of the electronic device 500 may determine whether a result of checking the fluctuations between the first parameter and the second parameter are within tolerances indicating availability for a determination of obstructive sleep apnea. That is, sometimes the data may be so out of threshold, that deviances must result from faulty performance of the test. In these cases, the determine of OSA is technically unavailable due to these external failures. Thus, for example, the processor 510 may determine whether the checked fluctuation result value (e.g., a value of “PPG_{Hjorth Mobility}”) is included in a given reference range. When the checked fluctuation result value is included in the given reference range, the processor 510 may determine that the first parameter and the second parameter are available for determination associated with obstructive sleep apnea. When the checked fluctuation result value is not included in the given reference range, the processor 510 may determine that the first parameter and the second parameter are unavailable for determination associated with obstructive sleep apnea. For example, data that are unavailable for determination associated with obstructive sleep apnea may be measured due to an error in measurement (e.g., a bad wearing state of the electronic device 500 is not worn properly, or if the user undergoes testing with bad posture that interferes with measurement).

According to an embodiment, in operation 1850, the processor 510 of the electronic device 500 may provide the determination result with regard to the obstructive sleep apnea through the user interface. For example, when the first parameter and the second parameter are unavailable for determination associated with obstructive sleep apnea, the processor 510 may prompt retesting through the user interface. When the first parameter and the second parameter are available for determination associated with obstructive sleep apnea, the processor 510 may provide the determination result associated with obstructive sleep apnea through the user interface based on the method described with reference to FIGS. 6 to 17.

It should be appreciated that certain embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

The expression “configured to” used in the disclosure may be exchanged with, for example, “suitable for”, “having the capacity to”, “designed to”, “adapted to”, “made to”, or “capable of” according to the situation. The term “configured to” may not necessarily imply “specifically designed to” in hardware. Alternatively, in some situations, the expression “device configured to” may mean that the device, together with other devices or components, “is able to”. For example, the phrase “processor adapted (or configured) to perform A, B, and C” may mean a dedicated processor (e.g. embedded processor) for performing the corresponding operations or a generic-purpose processor (e.g., central processing unit (CPU) or application processor (AP)) that can perform the corresponding operations by executing one or more software programs stored in a memory device (e.g., memory (130)).

The term “module” used in this disclosure may mean a unit including, for example, one or a combination of hardware, software, and firmware. The term “module” may be interchangeably used with other terms, for example, such as unit, logic, logical block, component, or circuit. The “module” may be the minimum unit, or a part thereof, of an integrally implemented component. The “module” may be the minimum unit, or a part thereof, for performing one or more functions. The “module” may be implemented mechanically or electronically. For example, according to the present disclosure, the “module” may include at least one of an application-specific integrated circuit (ASIC) chip, field-programmable gate arrays (FPGAs), and a programmable-logic device, which are known or to be developed later and perform particular functions.

According to certain embodiments of the disclosure, at least some of the devices (for example, modules or functions thereof) or the method (for example, operations) according to the present disclosure may be implemented by a command stored in a computer-readable storage medium in a programming module form. The instruction, when executed by a processor (e.g., the processor 120), may cause the one or more processors to execute the function corresponding to the instruction. The computer-readable storage medium may be, for example, the memory 130. The computer readable recoding medium may include a hard disk, a floppy disk, magnetic media (for example, a magnetic tape), optical media (for example, a compact disc read only Memory (CD-ROM) and a DVD), magneto-optical media (for example, a floptical disk), a hardware device (for example, a ROM, a RAM, a flash memory), and the like. In addition, the program instructions may include high class language codes, which can be executed in a computer by using an interpreter, as well as machine codes made by a compiler.

According to certain embodiments, each component (e.g., a module or a program) of the above-described components may include a single entity or multiple entities. According to certain embodiments, one or more of the above-described components may be omitted, or one or more other components may be added. Alternatively or additionally, a plurality of components (e.g., modules or programs) may be integrated into a single component. In such a case, according to certain embodiments, the integrated component may still perform one or more functions of each of the plurality of components in the same or similar manner as they are performed by a corresponding one of the plurality of components before the integration. According to certain embodiments, operations performed by the module, the program, or another component may be carried out sequentially, in parallel, repeatedly, or heuristically, or one or more of the operations may be executed in a different order or omitted, or one or more other operations may be added.

1. An electronic device, comprising:
a housing:
a display visible through a first portion of the housing;
a photoplethysmogram (PPG) sensor exposed through a second portion of the housing;
a memory disposed within the housing;
a processor disposed within the housing and operatively connected with the memory, the display and the PPG sensor,
wherein the memory stores instructions that, when executed, cause the processor to:
receive, for a user, first data from the PPG sensor, the first data including a pulse signal measured during a first period, and associated with a heartbeat and respiration of the user;
determine a first parameter associated with a high-frequency component of the pulse signal, and a second parameter associated with a low-frequency component of the pulse signal, based on at least a portion of the first data;
determine fluctuation ranges for the high-frequency component of the first parameter and the low-frequency component of the second parameter;
determine whether at least the determined fluctuation ranges are indicative of obstructive sleep apnea (OSA); and
provide information associated with the determination of whether the at least the determination fluctuation ranges are indicative of OSA through the display.

2. The electronic device of claim 1, wherein, when the instructions are executed, the processor is configured to:
classify the user into one of multiple OSA risk degrees using the first data based on at least a portion of the first parameter and the second parameter, the OSA risk degrees including a first severity, a second severity, or a third severity.

3. The electronic device of claim 2, wherein, when the instructions are executed, the processor is configured to:
receive basic biometric information of a user, the basic biometric information including one or more of age, gender, height, weight, neck circumference, waist circumference, or hip circumference; and
prior to receiving the first data, determine whether the user is classified into the first severity or the third severity, based on at least a portion of the received basic biometric information.

4. The electronic device of claim 2, further comprising:
a motion sensor,
wherein, when the instructions are executed, the processor is configured to:
receive second data from the PPG sensor and the motion sensor, wherein the second data includes respiration rate and movement of the user.

5. The electronic device of claim 4, wherein, when the instructions are executed, the processor is configured to:
determine an accuracy of the first data by comparing at least a portion of the first data and at least a portion of the second data; and
display guidance information on the display prompting reinput of the first data, based on the determined accuracy.

6. The electronic device of claim 4,
wherein, when the instructions are executed, the processor is configured to:
measure the respiration rate through the PPG sensor after displaying a prompt guiding the user through measurement of the respiration rate through the display; and
measure the movement through the PPG sensor and the motion sensor after displaying a prompt guiding the user through measurement of the movement through the display.

7. An electronic device comprising:
a housing;
a display viewable through a first portion of the housing;
a photoplethysmogram (PPG) sensor exposed through a second portion of the housing; and
a memory disposed within the housing, and
a processor disposed within the housing and operatively connected with the memory, the display and the PPG sensor,
wherein the memory stores instructions that, when executed, cause the processor to:
receive, from the PPG sensor, first data including a heart rate (HR) data of a user measured during a first period;
determine a parameter associated with a rate-of-change pattern included in the HR data, based on at least a portion of the first data;
determine whether at least a portion of the determined parameter is indicative of obstructive sleep apnea (OSA); and
provide information associated with the determination whether the at least the portion of the determined parameter is indicative of OSA through the display.

8. The electronic device of claim 7, further comprising:
a motion sensor,
wherein the instructions cause the processor to:
receive second data from the PPG sensor and the motion sensor, the second data including respiration data and movement data;
determine an accuracy of the first data, based on a comparison of at least a portion of the first data and at least a portion of the second data; and
display guidance information on the display prompting reinput of the first data, based on the determined accuracy.

9. The electronic device of claim 7, wherein the determined parameter includes a first parameter indicating a high-frequency component of the first data, and a second parameter indicating a low-frequency component of the first data, and
wherein, when the instructions are executed, the processor is configured to:
classify the first data as one of a first severity, a second severity, or a third severity based on at least a portion of the first parameter and the second parameter.

10. The electronic device of claim 9, wherein, when the instructions are executed, the processor is configured to:
receive basic biometric information of the user, the basic biometric information including one or more of age, gender, height, weight, neck circumference, waist circumference, or hip circumference; and
prior to receiving the HR data, determine whether the user is classified into the first severity or the third severity based on at least a portion of the basic biometric information.

11. The electronic device of claim 9, wherein, when the instructions are executed, the processor is configured to:
when the user is classified into the second severity, further classify the user into one of two sub-severities included within the second severity based on the second data.

12. The electronic device of claim 11, wherein, when the instructions are executed, the processor is configured to:
determine, based on a portion of the second data, a first sub-data including a change pattern in the HR data,
wherein the further classification of the user into one of the two sub-seventies is based on the first sub-data.

13. The electronic device of claim 12, wherein the first sub-data is determined based on a difference between a nonlinear regression line and a heart rate.

14. The electronic device of claim 12, wherein, when the instructions are executed, the processor is configured to:
determine a second sub-data including a rate of change in the HR data based on a portion of the second data; and
determine an accuracy of the further classification based on the second sub-data.

15. The electronic device of claim 14, wherein the second sub-data is determined by a difference between a maximum value of the HR data and a minimum value of the HR data.

16. A method in an electronic device is disclosed, including:
receiving, for a user, first data from a photoplethysmogram (PPG) sensor, the first data including a pulse signal measured during a first period, and associated with a heartbeat and respiration of the user; determining a first parameter associated with a high-frequency component of the pulse signal, and a second parameter associated with a low-frequency component of the pulse signal, based on at least a portion of the first data;
determining fluctuation ranges for the high-frequency component and the low-frequency component;
determining whether at least the determined fluctuation ranges are indicative of obstructive sleep apnea (OSA); and
displaying information associated with the determination of whether the at least the determined fluctuation ranges are indicative of OSA through the display.
